(19) United States
(12) Patent Application Publication
(10) Pub. No.: US 2025/0262045 A1
(43) Pub. Date: Aug. 21, 2025

(54) COMPOSITE DIFFRACTIVE MULTIFOCAL INTRAOCULAR LENS

(71) Applicant: (INDUSTRY-UNIVERSITY COOPERATION FOUNDATION HANYANG UNIVERSITY), Seoul (KR)

(72) Inventor: Seok Ho SONG, Seoul (KR)

(21) Appl. No.: 18/857,068

(22) PCT Filed: Apr. 20, 2023
(86) PCT No.: PCT/KR2023/005366

§ 371 Date: Dec. 27, 2024

(30) Foreign Application Priority Data
Apr. 21, 2022 (KR) 10-2022-0049579

(51) Int. Cl. A61F 2/16; G02B 3/10; G02B 27/42

(52) U.S. Cl. CPC A61F 2/1618; G02B 3/10; G02B 27/42

(57) ABSTRACT

The present invention relates to a composite diffractive intraocular lens having at least one optical magnification or focal length. The composite diffractive intraocular lens comprises and combines a first diffractive lens structure having a saw-tooth shape and an increasing phase change values in a plurality of regions divided in the radial direction from the center of the lens, and a second diffractive lens structure having a saw-tooth shape and a decreasing phase change values in the same regions as the plurality of regions, and has a composite phase profile structure in which phase values at the boundaries of the regions do not sharply increase or decrease vertically.

### TECHNICAL FIELD

Example embodiments relate to a combined diffractive multifocal intraocular lens, and more particularly, to a technical concept of combining a first diffractive lens structure with a sawtooth shape, configured to increase a phase change value, and a second diffractive lens structure with a sawtooth shape, configured to decrease a phase change value, to create a phase profile that gradually increases or decreases in a radial direction from the center of the lens.

### RELATED ART

Diffractive lenses with multifocal or multiple focal points are widely used in optical lenses, especially intraocular lenses (IOLs) for ophthalmic purposes, providing high-quality vision for both near and far objects simultaneously. These lenses are extensively utilized in ophthalmic treatments to offer enhanced vision correction.

For example, MF-IOLs (Multifocal Intraocular Lenses) with surfaces containing one or more trifocal diffraction patterns for near, intermediate, and far vision, and one or more bifocal diffraction patterns for near and far vision, have been commercialized.

To date, technologies for manufacturing commercially available multifocal diffractive lenses typically involve precisely processing the anterior or posterior surface of a monofocal lens into a surface-relief shape in the form of a Fresnel diffraction lens.

However, as the number of focal points in multifocal diffractive lenses increases, the complexity of the surface-relief shape required for the lens gradually increases, making MF-IOL production challenging. Incomplete manufacturing may lead to increased light scattering, causing visual disturbances such as blur, halo, and starburst effects.

### DETAILED DESCRIPTION

### Technical Subject

An objective of example embodiments is to provide a combined diffractive MF-IOL structure with a greatly simplified surface-relief shape that achieves vision correction effects similar to those of conventional MF-IOLs.

### Solution

According to an example embodiment, there is provided a combined diffractive multifocal intraocular lens including a first diffractive lens structure with a sawtooth shape, configured to increase a phase change value in each of multiple radial regions in a radial direction from a center of the lens, a second diffractive lens structure with a sawtooth shape, configured to decrease a phase change value in each of the multiple radial regions as the first diffractive lens structure, and wherein the first diffractive lens structure and the second diffractive lens structure are combined to form a combined phase profile structure, configured to prevent a phase value at the boundary between each of the multiple radial regions from increasing or decreasing abruptly in a vertical direction.

According to an example embodiment, there is provided a combined diffractive multifocal intraocular lens, wherein in odd-numbered regions in each of the multiple radial regions, the phase change value of the first diffractive lens structure is used, and in even-numbered regions, the phase change value of the second diffractive lens structure is used.

According to an example embodiment, there is provided a combined diffractive multifocal intraocular lens, wherein in even-numbered regions in each of the multiple regions, the phase change value of the first diffractive lens structure is used, and in odd-numbered regions, the phase change value of the second diffractive lens structure is used.

According to an example embodiment, there is provided a combined diffractive multifocal intraocular lens, wherein a light intensity value at each of the multiple foci generated by the combined phase profile structure is adjusted according to a magnitude of a maximum phase value.

According to an example embodiment, there is provided a combined diffractive multifocal intraocular lens, wherein a magnitude of a maximum phase value of the combined phase profile structure remains constant at a given distance in the radial direction from the center of the lens.

According to an example embodiment, there is provided a combined diffractive multifocal intraocular lens, wherein the maximum phase value of the combined phase profile structure decreases in the radial direction from the center of the lens.

According to an example embodiment, there is provided a combined diffractive multifocal intraocular lens, wherein the phase change values of the first diffractive lens structure and the second diffractive lens structure used in each of the multiple regions are configured to be adjusted and combined so that a maximum phase value of the combined phase profile decreases in the radial direction from the center of the lens.

According to an example embodiment, there is provided a combined diffractive multifocal intraocular lens, wherein a distance of a first region among the multiple regions is configured to set the phase value at a radial center of the combined phase profile to zero.

According to an example embodiment, there is provided a combined diffractive multifocal intraocular lens, wherein a distance of a first region among the multiple regions is configured to set the phase value at a radial center of the combined phase profile to be non-zero.

According to an example embodiment, there is provided a combined diffractive multifocal intraocular lens including a first diffractive lens structure with a sawtooth shape, configured to increase a positive phase value in each of multiple regions in a radial direction from the center of the lens, a second diffractive lens structure with a sawtooth shape, configured to decrease a negative phase value in each of the multiple regions, and wherein the first diffractive lens structure and the second diffractive lens structure are alternately combined to form a combined phase profile structure.

According to an example embodiment, there is provided a combined diffractive multifocal intraocular lens, wherein a maximum phase value of the first diffractive lens structure and a maximum phase value of the second diffractive lens structure are different from each other at a given radial distance in the radial direction from the center of the lens.

According to an example embodiment, there is provided a combined diffractive multifocal intraocular lens, wherein a portion where the phase value changes abruptly in a vertical direction at a boundary between each of the multiple regions is formed by a difference between a maximum phase value of the first diffractive lens structure and a maximum phase value of the second diffractive lens structure.

According to an example embodiment, there is provided a combined diffractive multifocal intraocular lens, wherein the combined phase profile structure is configured to adjust the light intensity at each of multiple foci generated according to the ratio of the maximum phase value of the first diffractive lens structure to the maximum phase value of the second diffractive lens structure.

### Effect

According to some example embodiments, it is possible to achieve very easy manufacturing due to a simplified surface-relief structure, significantly reducing light scattering and minimizing visual disturbances.

Additionally, according to some example embodiments, it is possible to create a combined diffractive MF-IOL structure with three or more focal points.

Further, according to some example embodiments, it is possible to adjust the light intensity at multiple focal positions by varying the maximum phase value as the radius of the lens increases.

Moreover, according to some example embodiments, it is possible to vary the distance from the central point of the lens to the first point where the phase value changes, depending on the intended use.

### BRIEF DESCRIPTION OF DRAWINGS

FIG. 1 is a cross-sectional view illustrating a conventional diffractive multifocal lens.

FIG. 2 is a diagram illustrating a cross-sectional view of a surface-relief shape of a conventional Fresnel diffraction lens.

FIG. 3 is a diagram illustrating various example embodiments of the phase profile of a conventional Fresnel diffraction lens structure.

FIG. 4 is a diagram illustrating light intensity-related data measured along the z-axis of a conventional Fresnel diffraction lens structure.

FIG. 5 is a diagram illustrating the phase profile of a combined diffractive multifocal intraocular lens structure according to the first example embodiment.

FIG. 6 is a diagram illustrating light intensity-related data measured along the z-axis of the combined diffractive multifocal intraocular lens structure according to the first example embodiment.

FIG. 7 is a diagram illustrating the ratio of light intensity at three focal points based on changes in the phase value in the combined diffractive multifocal intraocular lens structure according to the first example embodiment.

FIG. 8 is a diagram illustrating the phase profile of a combined diffractive multifocal intraocular lens structure according to the second example embodiment.

FIG. 9 is a diagram illustrating the phase profile of a combined diffractive multifocal intraocular lens structure according to the third example embodiment.

FIG. 10 is a diagram illustrating the phase profile of a combined diffractive multifocal intraocular lens structure according to the fourth example embodiment.

FIG. 11 is a diagram illustrating the phase profile of a combined diffractive multifocal intraocular lens structure according to the fifth example embodiment.

FIG. 12 is a diagram illustrating the light scatter phenomenon and the distribution of light intensity detected by the detector after incident light passes through the conventional Fresnel diffraction lens structure shown in FIG. 3.

FIG. 13 is a diagram illustrating the light scatter phenomenon and the distribution of light intensity detected by the detector after incident light passes through the combined diffractive multifocal intraocular lens structure according to an example embodiment.

FIG. 14 is a diagram illustrating the design structure and actual manufactured structure of a conventional Fresnel diffraction lens as shown in FIG. 3.

FIG. 15 is a diagram illustrating the design structure and actual manufactured structure of a combined diffractive multifocal intraocular lens according to an example embodiment.

### BEST MODE

Hereinafter, a detailed description of the combined diffractive multifocal intraocular lens according to a preferred embodiment will be provided with reference to the accompanying drawings. In this description, the same reference numerals are used for the same components, and repetitive descriptions as well as detailed explanations of known functions and configurations that may obscure the gist of the invention are omitted. The embodiments of the invention are provided to more fully explain the present invention to those of ordinary skill in the art. Therefore, the shapes and sizes of elements in the drawings may be exaggerated for clarity.

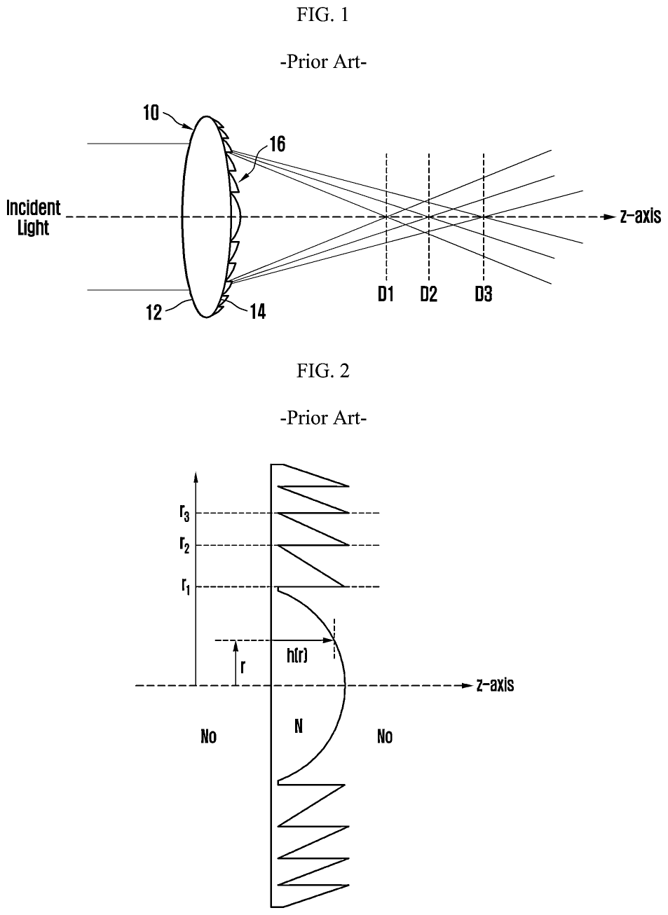

FIG. 1 is a cross-sectional view of a conventional diffractive multifocal lens, and FIG. 2 is a diagram showing a cross-sectional view of the surface-relief shape of a conventional Fresnel diffraction lens.

FIG. 1 is a cross-sectional view illustrating the optic part of a conventional diffractive multifocal lens (diffractive MF-IOL) or a diffractive multifocal intraocular lens.

As shown in FIG. 1, the optic part 10 of the lens includes a front surface 12 and a rear surface 14. On the surface of the rear surface 14, a diffractive surface profile with a surface-relief shape in the form of a Fresnel diffraction lens is formed. The diffractive surface profile may be formed on the rear surface, the front surface, or on both the front and rear surfaces of the lens.

Incident light along the z-axis direction passes sequentially through the front surface 12, the rear surface 14, and the diffractive surface profile 16, converging at multiple focal points. The positions where the focal points converge are referred to as focal planes, and in FIG. 1, three focal planes (D1, D2, D3) are shown as an example embodiment. The number of focal points or focal planes may vary from one to two or more, depending on the shape of the diffractive surface profile.

Meanwhile, according to a diffractive lens generally known as a Fresnel lens, as shown in FIG. 2, the diffractive lens has a sawtooth shape that increases in radius from the center of the lens, and the discontinuous surfaces at radius (r₁, r₂, . . . rⱼ) are defined by Equation 1 below.

$${r}_{j}^{2} = 2 j λ F \qquad [ Equation 1 ]$$

Here, rⱼ represents the j-th radius from the center of the diffractive lens, λ denotes the wavelength of the incident light, and F refers to the focal length from the center of the diffractive lens.

Light incident perpendicularly onto the diffractive lens surface along the z-axis direction passes through a thickness of h(r), after which the phase value Φ(r) according to the radius is calculated as shown in Equation 2 below.

$$Φ ( r ) = ( 2 π / λ ) \times h ( r ) \times \{ N - {N}_{0} \} \qquad [ Equation 2 ]$$

Here, N is the refractive index of the diffractive lens medium, and N₀ is the refractive index of the external medium.

Additionally, the phase distribution as shown in Equation 2 results in the formation of multiple focal points as the distance from the diffractive lens increases, as represented in Equation 3 below.

$${F}_{m} = \frac{F}{m} \qquad [ Equation 3 ]$$

In FIG. 1, the focal planes formed at intervals of Fₘ distance according to Equation 3 are labeled as D1, D2, and D3. Of course, as described above, the number of focal planes or focal points according to the diffraction order may vary depending on the shape of the diffractive surface profile.

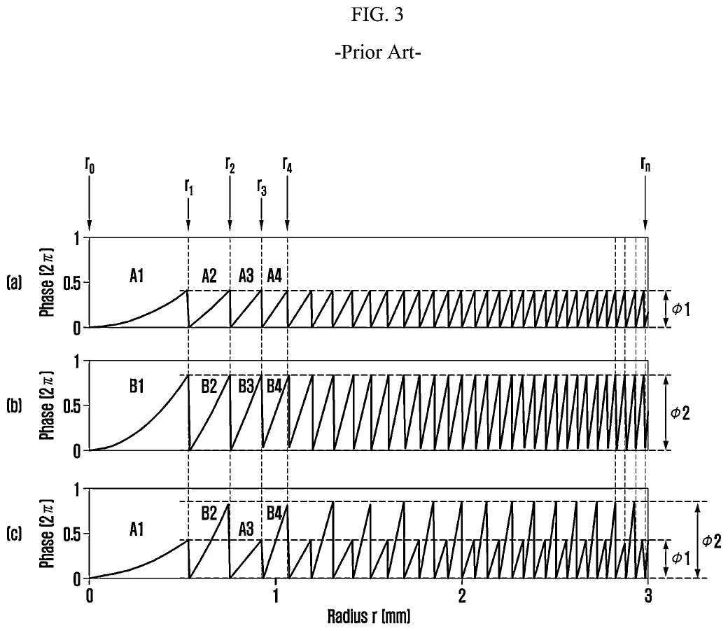

FIG. 3 is a diagram illustrating various example embodiments of the phase profile of a conventional Fresnel diffraction lens structure.

(a) to (c) of FIG. 3 show the phase distribution representing the phase difference of light rays passing through a radial distance r from the optical axis center (where r=0) in the transmitted wavefront incident on a conventional Fresnel diffraction lens (or intraocular lens) structure. The phase difference distribution shown in (a) to (c) of FIG. 3 is within the wavelength range of the incident light and has a sawtooth-shaped cross-section.

Specifically, the conventional Fresnel diffraction lens structures shown in (a) and (b) of FIG. 3 each have a sawtooth-shaped phase structure with an increasing phase change value in multiple regions segmented from the center of the lens. In the Fresnel diffraction lens structure shown in (a) of FIG. 3, the maximum phase value is Φ1, while in (b) of FIG. 3, it is Φ2, where Φ2 is greater than Φ1.

More specifically, in (a) and (b) of FIG. 3, the phase profile is divided into multiple regions in a radial direction from the center of the lens (where r=0), with each boundary between regions determined by radial values rⱼ, forming discontinuous surfaces. These multiple regions may be divided as follows: the region from the center r=0 to r₁ (A1 region in (a) of FIG. 3, B1 region in (b) of FIG. 3), the region from r₁ to r₂ (A2 region in (a) of FIG. 3, B2 region in (b) of FIG. 3), and so on. The boundary between the A1 and A2 regions (or the boundary between the B1 and B2 regions) is determined by r₁, while the boundary between the A2 and A3 regions is determined by r₂ (or the boundary between the B2 and B3 regions). The rⱼ values, which are boundary points of each region, may be calculated by Equation 1. Therefore, if the wavelength of the incident light is the same, the multiple regions segmented in (a) and (b) of FIG. 3 are identical to each other.

The phase value in the Fresnel diffraction lens structure may vary between 0 and 2π, and the maximum value may be appropriately selected within this range depending on the intended use of the lens. However, in the Fresnel diffraction lens structure, the maximum phase value may increase by multiples of 2π, and within this maximum phase value range, a lens with an appropriate phase change may be selected according to its intended purpose. This configuration may also be applied to the example embodiment.

The conventional Fresnel diffraction lens structures shown in (a) and (b) of FIG. 3 exhibit a sawtooth shape where the phase value starts at 0 at the beginning of each region and increases to the boundary of the neighboring region as the radius increases, then drops vertically to 0 at the boundary. This phase change repeats in each region, forming a sawtooth shape. The phase value Φ(r) according to the radius may be calculated by Equation 2.

Meanwhile, in the conventional Fresnel diffraction lens structures shown in (a) and (b) of FIG. 3, the maximum phase values in each region are Φ1 and Φ2, respectively, and are constant. In other words, as shown in (a) and (b) of FIG. 3, the phase change range in each region of the conventional Fresnel diffraction lens increases radially from the lens center, consistently repeating from 0 to Φ1 and 0 to Φ2.

The phase profile of the conventional Fresnel diffraction lens structure shown in (c) of FIG. 3 has a combined diffraction profile, where the conventional Fresnel diffraction lens structures shown in (a) and (b) of FIG. 3 are alternately combined in each region.

Specifically, in the combined diffraction profile shown in (c) of FIG. 3, the phase distribution in the A1 region of (a) of FIG. 3 is used for the first region (from r=0 to r=r₁) relative to the radial center of the lens, and the phase distribution in the B2 region of (b) of FIG. 3 is used for the second region (from r=r₁ to r=r₂). The phase distributions in subsequent regions may be understood with reference to FIG. 3.

Meanwhile, the conventional Fresnel diffraction lens structures shown in (a) and (b) of FIG. 3 each have a sawtooth shape in which the phase value in each region consistently increases. Therefore, even when the Fresnel diffraction lens structures shown in (a) of FIG. 3 and (b) of FIG. 3 are alternately combined in the composite diffraction profile structure shown in (c) of FIG. 3, the phase value decreases vertically at the boundary between neighboring regions.

As described above, in the composite diffraction profile structure of (c) of FIG. 3, the Fresnel diffraction lens structure of (a) of FIG. 3 used in the A1 region increases the phase value to Φ1, then decreases vertically at r₁, while the Fresnel diffraction lens structure of (b) of FIG. 3 used in the A2 region increases the phase value to Φ2, then decreases vertically at r₂.

Meanwhile, in another embodiment, two conventional Fresnel diffraction lens structures may have the same maximum phase change range (Φ1=Φ2), and a composite diffraction profile may be created using these two lens structures.

Additionally, while the phase profiles shown in (a) and (b) of FIG. 3 have an increasing phase value in each region, in another embodiment, the phase value in each region may decrease, and a composite diffraction profile may be formed by combining these two lens structures.

However, even in composite diffraction profile structures where each Fresnel diffraction lens structure is combined according to other embodiments, the phase value at the boundary points (r₁, r₂, etc.) changes vertically.

The reason for using the composite diffraction profile shown in (c) of FIG. 3 is that it has the advantage of increasing the number of focal points and more diversely adjusting the light intensity distribution at multiple focal points, compared to the Fresnel diffraction lenses shown in (a) and (b) of FIG. 3.

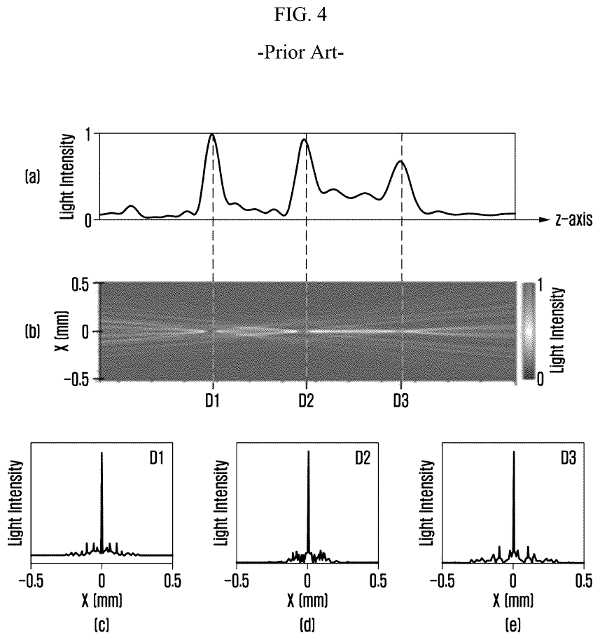

FIG. 4 is a diagram illustrating light intensity-related data measured along the z-axis of a conventional Fresnel diffraction lens structure, with reference to FIG. 3.

FIG. 4 shows the results of calculating three focal points in a diffractive lens with the combined diffraction profile shown in (c) of FIG. 3, where Φ1=2π×0.4, Φ2=2π×0.8.

Specifically, (a) of FIG. 4 shows the distribution of light intensity measured along the z-axis after incident light has passed through the diffractive lens structure with the combined diffraction profile shown in (c) of FIG. 3. In (a) of FIG. 4, an example embodiment with three focal planes (D1, D2, and D3) is illustrated. The light intensity values at the focal planes D1 and D2 are nearly similar, while the light intensity at the focal plane D3 is lower than those at the focal planes D1 and D2.

Meanwhile, in the combined diffraction profile structure shown in (c) of FIG. 3, the distribution of light intensity may vary depending on the magnitude of Φ1 and/or Φ2.

(b) of FIG. 4 illustrates the process in which light incident on the lens with the combined diffraction profile structure shown in (c) of FIG. 3 propagates along the z-axis and converges at the focal planes D1, D2, and D3. As shown in (b) of FIG. 4, the light intensity at D1, D2, and D3 is greater than at other positions.

(c) to (e) of FIG. 4 respectively show the distribution of light intensity along the x-axis at the focal planes D1, D2, and D3. As shown in (c) to (e) of FIG. 4, each focal point (D1, D2, D3) exhibits the highest light intensity at the focal center (X=0 mm). The distribution of light intensity around the focal center (X=0 mm) is narrow, and the greater the light intensity at the focal center (X=0 mm), the sharper the resulting image.

Meanwhile, as described above, the conventional multifocal lens (or intraocular lens) shown in FIG. 3 has a shape in which the phase value changes abruptly in a vertical direction at each radial point (r₁, r₂, . . . rⱼ). The surface relief (surface profile) of the diffractive lens, which is formed in proportion to this phase value, must also be manufactured with an abrupt vertical shape. Additionally, as shown in (c) of FIG. 3, if there is a difference between the phase values Φ1 and Φ2 of the conventional Fresnel diffraction lens structures shown in (a) and (b) of FIG. 3 required for the combination, the manufacturing process becomes more complex, and the manufacturing difficulty increases as the radius increases.

If the surface relief shape of the diffractive lens is not properly formed vertically at each radial point but instead is manufactured with an inclined slope, the light intensity distribution shown in (c) to (e) of FIG. 4 will exhibit increased light intensity at areas other than X=0 mm, resulting in a failure to form a sharp focus.

In other words, the conventional diffractive multifocal lens (or intraocular lens) has caused various problems, such as blurred images or unclear vision. These issues mainly arise due to the limitations of manufacturing a vertically abrupt surface relief shape as shown in FIG. 3.

According to an example embodiment, a new design method for a combined diffraction profile is proposed to overcome the manufacturing limitations of the conventional diffractive multifocal lens (or intraocular lens).

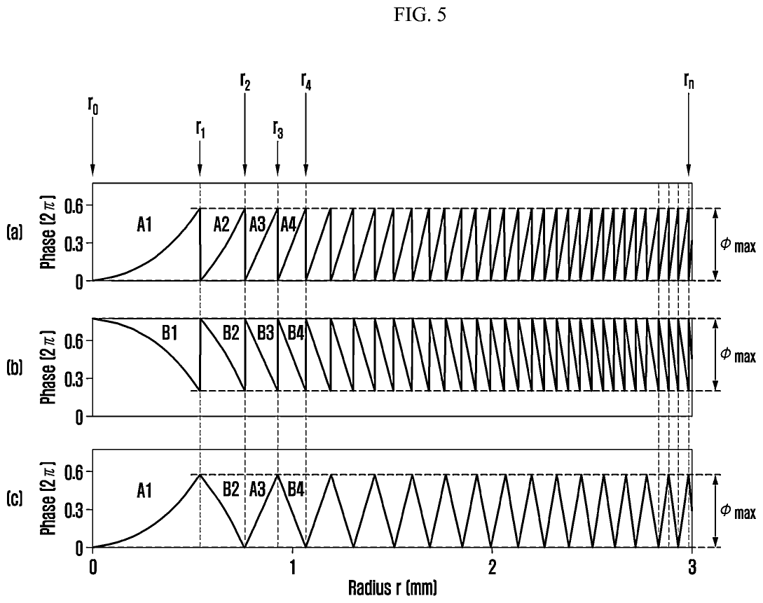

FIG. 5 is a diagram illustrating the phase profile of a combined diffractive multifocal intraocular lens structure according to the first example embodiment.

The phase profile of the combined diffractive multifocal intraocular lens according to the first example embodiment, as shown in (c) of FIG. 5, may be formed by combining the diffractive lens structures shown in (a) and (b) of FIG. 5. This phase profile formed in such a manner is referred to as a combined phase profile structure.

The diffractive lens structure shown in (a) of FIG. 5 has a sawtooth-shaped phase structure with an increasing phase change value in each of multiple regions segmented from the center of the lens. The diffractive lens structure shown in (b) of FIG. 5 has a sawtooth-shaped phase structure with a decreasing phase change value in each of multiple regions segmented from the center of the lens.

The segmentation of each region (A1, A2, . . . , B1, B2, . . . ) in FIG. 5, the phase value changes, and the formula for calculating the phase value may be referenced from the above description.

That is, the diffractive lens structure shown in (a) of FIG. 5 has a direction in which the phase value increases in each region (A1, A2, . . . ), while the diffractive lens structure shown in (b) of FIG. 5 has a direction in which the phase value decreases in each region (B1, B2, . . . ). Here, the phase distribution in (a) of FIG. 5 is referred to as a positive diffractive lens structure, and the phase distribution in (b) of FIG. 5 as a negative diffractive lens structure.

Meanwhile, in the diffractive lens structures shown in (a) and (b) of FIG. 5, the maximum phase value (Φmax) is the same, and the phase change range in each region of the diffractive lens remains constant from 0 to Φmax as it increases radially from the lens center.

The combined diffraction profile shown in (c) of FIG. 5 uses the phase distribution in the A1 region of (a) of FIG. 5 for the first region (from r=0 to r=r₁), and the phase distribution in the B2 region of (b) of FIG. 5 for the second region (from r=r₁ to r=r₂). The phase distribution for subsequent regions may be understood with reference to FIG. 5.

As shown in (c) of FIG. 5, the combined diffraction profile structure according to one example embodiment is a structure in which the positive diffractive lens structure (see (a) of FIG. 5) and the negative diffractive lens structure (see (b) of FIG. 5) are alternately combined.

By alternately combining the positive and negative diffractive lens structures in this way, the phase value at the boundary between neighboring regions does not decrease abruptly but changes gradually. Specifically, in (c) of FIG. 5, the phase value gradually increases in the A1 region and gradually decreases in the B2 region, with r₁ as the boundary. This phase value change repeats in subsequent regions.

This combined diffraction profile structure according to one example embodiment has a constant maximum phase height (Φmax) and allows more spacing between the repetitive relief structures. In other words, unlike conventional diffractive multifocal lens design techniques, it does not have an abrupt vertical decrease in surface relief shape at radial points (r₁, r₂, . . . ), making it easier to manufacture due to the constant height of the relief shape. This also addresses issues in conventional technology where images appear blurred or unclear.

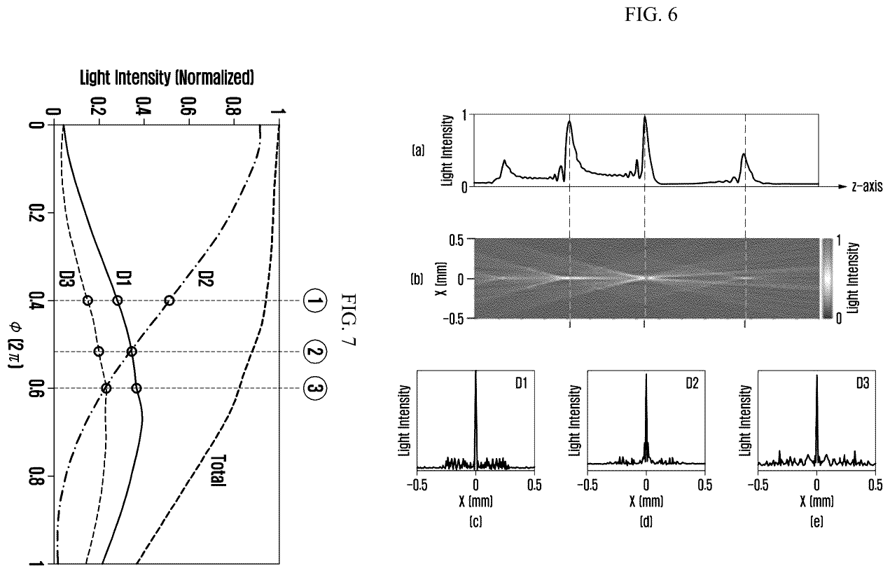

FIG. 6 is a diagram illustrating light intensity-related data measured along the z-axis of the combined diffractive multifocal intraocular lens structure according to the first example embodiment.

In (c) of FIG. 6, the results of calculating three focal points in a diffractive lens with the combined diffraction profile shown in (c) of FIG. 5 are displayed, where the maximum phase value Φmax=2π×0.6.

Specifically, FIG. 6(a) shows the distribution of light intensity measured along the z-axis after the incident light passes through the diffractive lens structure with the combined diffraction profile shown in (c) of FIG. 5. In FIG. 6(a), an example embodiment is illustrated in which three focal planes (D1, D2, D3) are formed. At this time, the light intensity values at the focal planes D1 and D2 are almost similar, while the light intensity at the focal plane D3 is lower than that at the focal planes D1 and D2.

Meanwhile, in the combined diffraction profile structure shown in (c) of FIG. 5, the distribution of light intensity may vary depending on the magnitude of the Φ value.

(b) of FIG. 6 illustrates the process in which light incident on the lens with the combined diffraction profile structure shown in (c) of FIG. 5 propagates along the z-axis and converges at the focal planes D1, D2, and D3. As shown in (b) of FIG. 6, the light intensity at points D1, D2, and D3 is greater than at other points.

(c) to (e) of FIG. 6 respectively show the distribution of light intensity along the x-axis at the focal planes D1, D2, and D3. From the light intensity distribution shown in (c) to (e) of FIG. 6, it may be seen that a very sharp focus converges at the point where X=0 mm. As described above, each focal point (D1, D2, D3) exhibits the highest light intensity at the focal center (X=0 mm). The light intensity distribution around the focal center (X=0 mm) is narrow, and the higher the light intensity at the focal center (X=0 mm), the clearer the resulting image appears.

Despite being significantly different from the conventional combined diffraction profile (see FIG. 3), the new structure of the combined diffraction profile according to one example embodiment exhibits multifocal formation results with very similar characteristics, as may be seen from the comparison results shown in FIGS. 4 and 6.

In summary, the lens with the combined diffraction profile according to one example embodiment has performance similar to that of the conventional combined diffractive lens, while offering the advantage of more easily enabling shape fabrication.

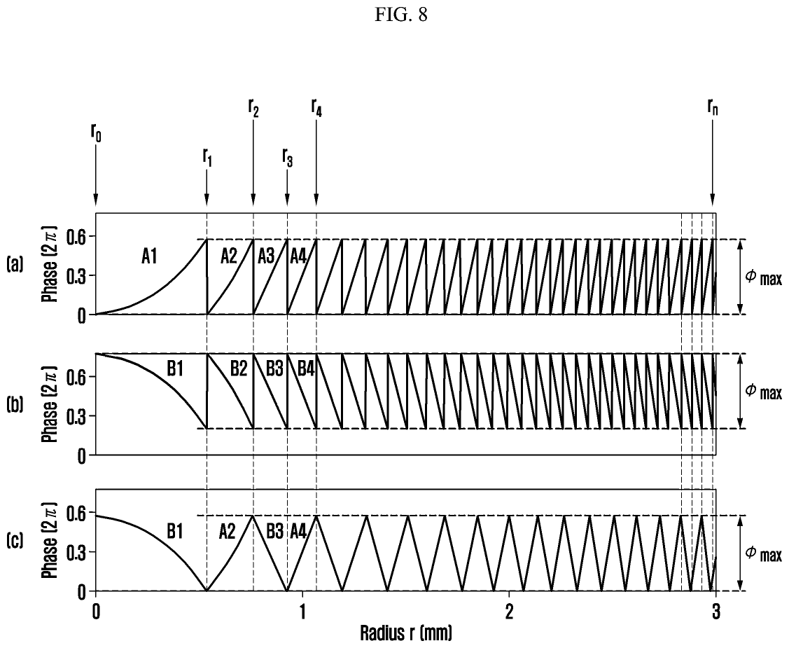

FIG. 7 is a diagram illustrating the ratio of light intensity at three focal points based on changes in the phase value in the combined diffractive multifocal intraocular lens structure according to the first example embodiment.

FIG. 7 illustrates the characteristics of light intensity formed at the three focal planes (D1, D2, D3) shown in FIG. 6, when the phase value Φ varies within the range of 0 to 2π. Here, when the maximum phase value Φmax=2π×0.4, the light intensity ratio of the three focal points is approximately D1:D2:D3=2:3:1. When Φmax=2π×0.52, the light intensity ratio of the three focal points is approximately D1:D2:D3=2:2:1, and when Φmax=2π×0.6, the light intensity ratio of the three focal points is approximately D1:D2:D3=1.5:1:1. In other words, by adjusting the Φmax value, it is possible to arbitrarily control the light intensity formed at multiple focal points.

FIG. 8 is a diagram illustrating the phase profile of a combined diffractive multifocal intraocular lens structure according to the second example embodiment.

The diffractive lens structures shown in (a) and (b) of FIG. 8 are identical to the diffractive lens structures shown in (a) and (b) of FIG. 6, respectively. However, unlike the combined diffraction shape shown in (c) of FIG. 6, the combined diffraction shape shown in (c) of FIG. 8 uses the B1 region shown in (b) of FIG. 8 as the first region and the A2 region shown in (a) of FIG. 8 as the second region. In other words, unlike in (c) of FIG. 6, in the second example embodiment of the example embodiment, the combination order is reversed.

Even with this combination, the phase value at the boundary between neighboring regions does not decrease abruptly but changes gradually, as described in the first example embodiment of the example embodiment above. In other words, only the direction of the phase change is reversed, and the effect produced, as compared to the conventional combined diffraction profile, is as described above. Additionally, in the case of a lens manufactured according to the second example embodiment of the example embodiment, data such as the distribution of light intensity at each focal point (D1, D2, D3) as explained in the first example embodiment may be obtained in nearly the same manner (see FIGS. 6 and 7).

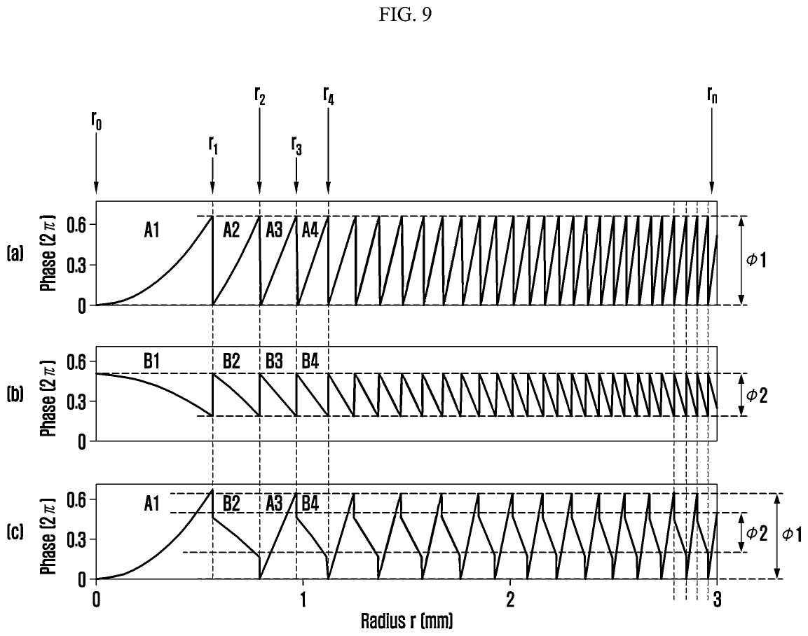

FIG. 9 is a diagram illustrating the phase profile of a combined diffractive multifocal intraocular lens structure according to the third example embodiment.

The diffractive lens structures shown in (a) and (b) of FIG. 9 have different maximum phase values (Φ1, Φ2) from each other. Among the two diffractive lens structures shown in (a) and (b) of FIG. 9, the first region of the combined profile shown in (c) of FIG. 9 uses the A1 region of (a) of FIG. 9, and the second region uses the B2 region of (b) of FIG. 9. However, due to the difference in the maximum phase values of the two diffractive lenses (Φ1−Φ2), a portion where the phase value changes abruptly at certain radial points appears.

Nonetheless, in the third example embodiment of the example embodiment, even if there is a portion where the phase value changes abruptly, the vertically cut sections are significantly reduced compared to a lens structure with a conventional combined diffraction profile, making the manufacturing process easier.

According to the third example embodiment of the example embodiment, by adjusting the ratio of the maximum phase values (Φ1, Φ2) of the two diffractive lenses, the light intensity at each focal point (D1, D2, D3) may be adjusted arbitrarily. In the example embodiment, the light intensity at each focal point (D1, D2, D3) is proportional to sin² (Φmax) with respect to the maximum phase value (Φmax). In the third example embodiment of the example embodiment, the light intensity at each focal point (D1, D2, D3) is adjusted according to the ratio of Φ1 to Φ2, more specifically according to the ratio of sin² (Φ1) to sin² (Φ2). Meanwhile, the light intensity at point D2 may be the remaining light intensity value after subtracting the light intensities at points D1 and D3.

In another embodiment of the example embodiment, among the two diffractive lens structures shown in (a) and (b) of FIG. 9, the first region of the combined profile shown in (c) of FIG. 9 may use the B1 region of (b) of FIG. 9, and the second region may use the A2 region of (a) of FIG. 9.

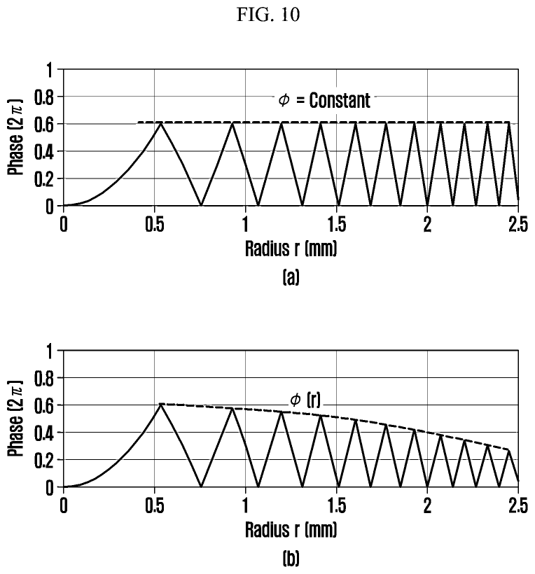

FIG. 10 is a diagram illustrating the phase profile of a combined diffractive multifocal intraocular lens structure according to the fourth example embodiment.

(a) of FIG. 10 shows a phase profile in which the maximum phase value remains constant in the radial direction from the center of a lens with a combined diffraction profile, and (b) of FIG. 10 shows a phase profile in which the maximum phase value (r) gradually decreases in the radial direction from the center of a lens with a combined diffraction profile according to the fourth example embodiment of the example embodiment. This type of phase profile may be referred to as an apodized shape. Meanwhile, in another embodiment of the example embodiment, the phase profile may have a reverse-apodized shape, in which the maximum phase value Φ(r) increases in the radial direction from the center of the lens, or an uneven shape, where the phase increase and decrease magnitudes differ.

The rate at which the maximum phase value decreases in the combined diffraction profile shown in (b) of FIG. 10 may be arbitrarily set depending on the case. For example, the maximum phase value may decrease by reducing the portion of the phase value that is high in each region, by maintaining the portion with a high phase value while increasing the portion with a low phase value, or by simultaneously changing both the high and low portions of the phase value, allowing the overall change magnitude to gradually decrease as the radius increases.

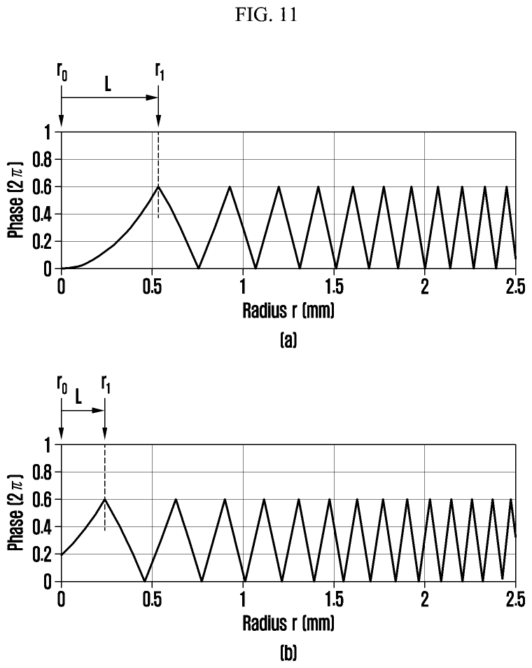

FIG. 11 is a diagram illustrating the phase profile of a combined diffractive multifocal intraocular lens structure according to the fifth example embodiment.

FIG. 11 illustrates an example embodiment in which the length (L) from the center point at r=0 to r1 of a lens with a combined diffraction profile may be arbitrarily varied according to the design. Depending on the change in the L value, the phase value at the center point r=0 may be 0 (see (a) of FIG. 11) or may be greater than 0 (see (b) of FIG. 11). The change in the L value may serve as a useful variable for adjusting the intensity ratio of multifocal light.

Meanwhile, in another embodiment of the example embodiment, it is also possible to combine three or more diffractive lens structures, and in this case, it is preferable to combine them so that the phase change gradually varies at the boundary of each region based on the radial points.

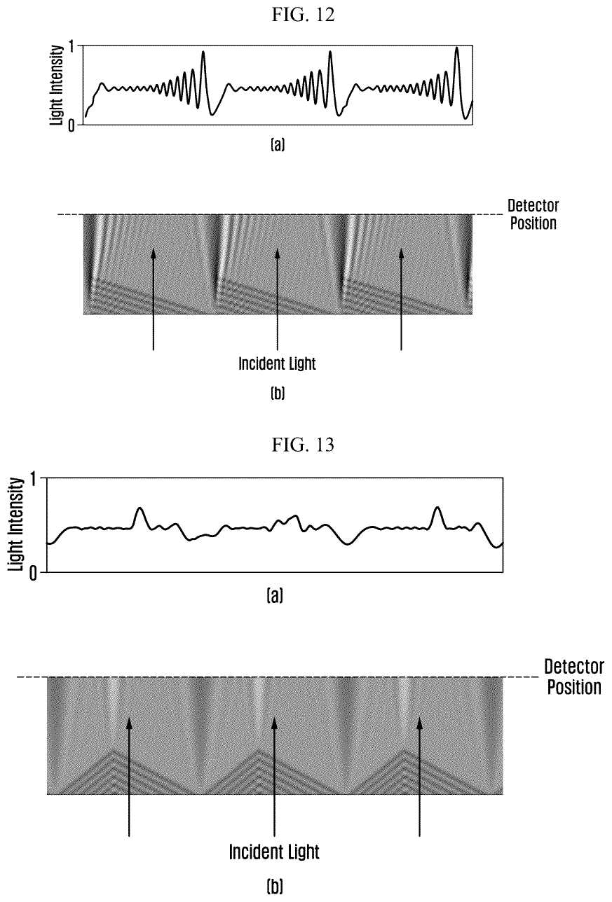

FIG. 12 is a diagram illustrating the light scatter phenomenon and the distribution of light intensity detected by the detector after incident light passes through the conventional Fresnel diffraction lens structure shown in FIG. 3. FIG. 12 shows the results of an experiment conducted using a lens manufactured according to the design of a conventional combined diffractive shape (see (c) of FIG. 3).

When incident light enters through a conventional lens shown in (b) of FIG. 12, the change in light intensity at each position is represented by the graph shown in (a) of FIG. 12. The range of variation in the light intensity graph indicates the degree of light dispersion. In the light intensity graph measured in (a) of FIG. 12, the variation range is approximately 2.8.

FIG. 13 is a diagram illustrating the light scatter phenomenon and the distribution of light intensity detected by the detector after incident light passes through the combined diffractive multifocal intraocular lens structure according to an example embodiment. An experiment was conducted using a lens manufactured according to the lens structure design with a combined diffractive shape according to the example embodiment shown in FIG. 13 (see (c) of FIG. 5).

When incident light enters through a lens with a combined diffraction profile according to the example embodiment shown in (b) of FIG. 13, the change in light intensity at each position is represented by the graph shown in (a) of FIG. 13.

In the light intensity graph measured in (a) of FIG. 13, the variation range is approximately 1.4, which, when compared with the variation range of the light intensity graph measured in (a) of FIG. 12, shows that the degree of light dispersion is reduced by about half.

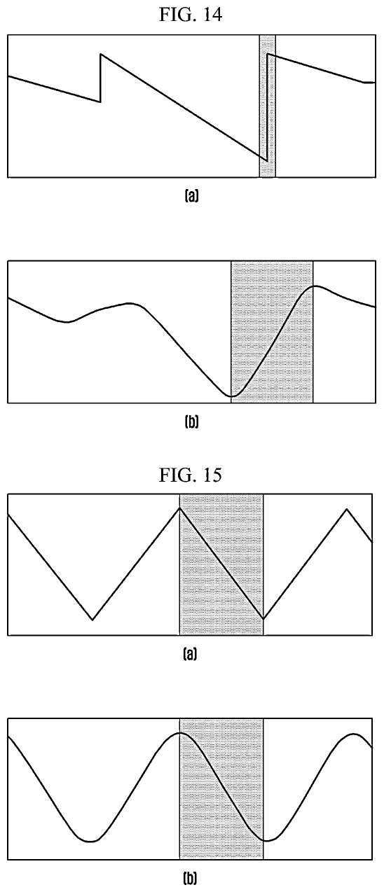

FIG. 14 is a diagram illustrating the design structure and the actual manufactured structure of the conventional Fresnel diffraction lens shown in FIG. 3, and FIG. 15 is a diagram illustrating the design structure and the actual manufactured structure of the combined diffractive multifocal intraocular lens according to the example embodiment.

In general, the fine sawtooth structure on the surface of a lens is manufactured by cutting the lens surface using a precision lathe tool. Since the cutting edge of the tool used in a precision lathe tool has a certain area, it is difficult to create very sharp portions.

(a) of FIG. 14 shows the design structure of a lens with a conventional combined diffraction profile, with a vertical cross-section formed on the lens surface. When cutting with a precision lathe tool according to this design structure, a structure like that shown in (b) of FIG. 14 is produced. In other words, although the vertical cross-section is intended to be cut according to the design structure, in reality, the vertical section becomes inclined.

(a) of FIG. 15 shows the design structure of a lens with a combined diffraction profile according to the example embodiment, in which only inclined cross-sections are formed on the lens surface, with no vertical cross-sections. When cutting with a precision lathe tool according to this design structure, a structure like that shown in (b) of FIG. 15 is produced. As shown in (b) of FIG. 15, according to an example embodiment, the inclined portions are manufactured to closely match the design structure.

In summary, when comparing the manufactured structures shown in FIGS. 14 and 15, both cases result in rounded ends at sharp points, but according to an example embodiment, the structure is manufactured to more closely match the design.

Meanwhile, according to an example embodiment, when cutting the lens surface using a precision lathe or similar tool, adjusting the cutting depth allows the phase value to be adjusted proportionally, enabling the manufacture of lenses with various phase distributions.

Although the example embodiment has been described with reference to an exemplary embodiment shown in the accompanying drawings, it is merely illustrative, and it will be understood by those skilled in the art that various modifications and equivalent other embodiments are possible. Therefore, the true scope of protection of the example embodiment should be defined solely by the appended claims.

What is claimed is:

1. A combined diffractive multifocal intraocular lens comprising:
a first diffractive lens structure with a sawtooth shape, configured to increase a phase change value in each of multiple regions in a radial direction from a center of the lens;
a second diffractive lens structure with a sawtooth shape, configured to decrease a phase change value that decreases in each of the multiple regions as the first diffractive lens structure; and
wherein the first diffractive lens structure and the second diffractive lens structure are combined to form a combined phase profile structure, configured to prevent a phase value at a boundary between each of the multiple regions from increasing or decreasing abruptly in a vertical direction.

2. The combined diffractive multifocal intraocular lens of claim 1, wherein in odd-numbered regions in each of the multiple regions are configured to use the phase change value of the first diffractive lens structure, and in even-numbered regions are configured to use the phase change value of the second diffractive lens structure.

3. The combined diffractive multifocal intraocular lens of claim 1, wherein in even-numbered regions in each of the multiple regions are configured to use the phase change value of the first diffractive lens structure, and in odd-numbered regions are configured to use the phase change value of the second diffractive lens structure.

4. The combined diffractive multifocal intraocular lens of claim 1, wherein the combined phase profile structure is configured to adjust a light intensity value at each of multiple foci according to a magnitude of a maximum phase value.

5. The combined diffractive multifocal intraocular lens of claim 1, wherein the combined phase profile structure is configured to maintain a constant magnitude of a maximum phase value at a given distance in the radial direction from the center of the lens.

6. The combined diffractive multifocal intraocular lens of claim 1, wherein the combined phase profile structure is configured to decrease a maximum phase value in the radial direction from the center of the lens.

7. The combined diffractive multifocal intraocular lens of claim 6, wherein the phase change values of the first diffractive lens structure and the second diffractive lens structure used in each of the multiple regions are configured to be adjusted and combined so that a maximum phase value of the combined phase profile decreases in the radial direction from the center of the lens.

8. The combined diffractive multifocal intraocular lens of claim 1, wherein a distance of a first region among the multiple regions is configured to set the phase value at a radial center of the combined phase profile to zero.

9. The combined diffractive multifocal intraocular lens of claim 1, wherein a distance of a first region among the multiple regions is configured to set the phase value at a radial center of the combined phase profile to be non-zero.

10. A combined diffractive multifocal intraocular lens comprising:
a first diffractive lens structure with a sawtooth shape, configured to increase a positive phase value in each of multiple regions in a radial direction from the center of the lens;
a second diffractive lens structure with a sawtooth shape, configured to decrease a negative phase value in each of the multiple regions; and
wherein the first diffractive lens structure and the second diffractive lens structure are alternately combined to form a combined phase profile structure.

11. The combined diffractive multifocal intraocular lens of claim 10, wherein a maximum phase value of the first diffractive lens structure and a maximum phase value of the second diffractive lens structure are different from each other at a given radial distance in the radial direction from the center of the lens.

12. The combined diffractive multifocal intraocular lens of claim 11, wherein a portion where the phase value changes abruptly in a vertical direction at a boundary between each of the multiple regions is formed by a difference between a maximum phase value of the first diffractive lens structure and a maximum phase value of the second diffractive lens structure.

13. The combined diffractive multifocal intraocular lens of claim 11, wherein the combined phase profile structure is configured to adjust a light intensity at each of multiple foci generated according to a ratio of the maximum phase value of the first diffractive lens structure to the maximum phase value of the second diffractive lens structure.
